(19) United States
(12) Patent Application Publication
(10) Pub. No.: US 2020/0253552 A1
(43) Pub. Date: Aug. 13, 2020

(54) WEARABLE COGNITIVE FUNCTION EVALUATION ASSISTING DEVICE

(71) Applicant: Han-Wei Zhang, New Taipei City (TW)

(72) Inventor: Han-Wei Zhang, New Taipei City (TW)

(21) Appl. No.: 16/862,861

(22) Filed: Apr. 30, 2020

Related U.S. Application Data
(63) Continuation-in-part of application No. 14/607,148, filed on Jan. 28, 2015, now abandoned.

(30) Foreign Application Priority Data
Jan. 29, 2014 (TW) 103202095

(51) Int. Cl. A61B 5/00; A61B 5/1455

(52) U.S. Cl. CPC A61B 5/6804; A61B 5/6805; A61B 5/4803; A61B 2562/06; A61B 5/0004; A61B 5/002; A61B 5/4088; A61B 5/14552

(57) ABSTRACT

A wearable cognitive function evaluation assisting device includes a wearable garment, a near infrared (NIR) light source, an image capturing unit, a storage unit, a projection unit, a sound-recording unit, a processing unit, a transceiver, a control unit, and a power supply unit. The image capturing unit can be selectively extracted from the wearable garment for capturing a continuous image of an oxygenated heme (HbO₂) where the NIR light source is irradiated. The projection unit can be selectively extracted from the wearable garment for projecting one of the cognitive environment images stored in the storage unit. The processing unit converts the question and answer recorded by sound-recording unit into a question and answer audio signal. The transceiver receives the continuous image of HbO₂ and the question and answer audio signal and then transmits them out.

### CROSS-REFERENCES TO RELATED APPLICATIONS

This application is a continuation-in-part (CIP) of application Ser. No. 14/607,148, filed on Jan. 28, 2015 with claiming foreign priority of TW103202095.

This non-provisional application claims priority under 35 U.S.C. § 119(a) on Patent Application No. 103202095 filed in Taiwan, R.O.C. on 2014 Jan. 29, the entire contents of which are hereby incorporated by reference.

### BACKGROUND

### Technical Field

The instant disclosure relates to a wearable device, in particular, for cognitive function evaluation applications.

### Related Art

The brain cells changes maybe affect decline of cognitive functions. With the aging of the social population, the decline of cognitive functions of the elderly population becomes more and more common. Traditionally, the judgment of whether cognitive function is degraded includes the test of physiological changes and the test of behavioral ability.

The test of physiological changes mainly needs to capture the continuous image of oxyhemoglobin (HbO₂) to determine the oxygenation and oxygen consumption of his/her red blood cells. It is usually necessary to apply a developer into his/her body and performed on MRI. Such a test is uncomfortable due to the developer and it may be rejected.

The test of behavioral ability is usually judged by situational interviews or reactions of various picture cards, but this is not completely accurate, and it must still be comprehensive evaluated with the result of test of physiological change.

### SUMMARY

In view of the foregoing, the instant disclosure provides a wearable cognitive function evaluation assisting device. The wearable cognitive function evaluation assisting device includes a wearable garment, a near infrared (NIR) light source, an image capturing unit, a storage unit, a projection unit, a sound-recording unit, a processing unit, a transceiver, a control unit, and a power supply unit.

The near infrared (NIR) light source is disposed on the wearable garment, and can be selectively extracted from the wearable garment. The image capturing unit is disposed on the wearable garment and can be selectively extracted from the wearable garment for capturing a continuous image of an oxyhemoglobin (HbO₂) where the NIR light source is irradiated. The storage unit is disposed in the wearable garment and stores a plurality of cognitive environment images. The projection unit is disposed on the wearable garment and electrically connected to the storage unit, and can be selectively extracted from the wearable garment for projecting one of the cognitive environment images.

The sound-recording unit is disposed on the wearable garment and electrically connected to the storage unit for recording a question and answering sound. The processing unit is disposed in the wearable garment and electrically connected to the sound-recording unit for converting the question and answer sound into a question and answer audio signal and sending the question and answer audio signal out. The transceiver is disposed in wearable garment and electrically connected to the image capturing unit and the processing unit, the transceiver receives the continuous image of HbO₂ and the question and answer audio signal and then transmits the continuous image of HbO₂ and the question and answer audio signal out. The control unit is disposed on the wearable garment and electrically connected to turning on and off the NIR light source, the image capturing unit, the projection unit, and the sound-recording unit. The power supply unit supplies power to the NIR light source, the image capturing unit, the storage unit, the projection unit, the sound-recording unit, the processing unit, the transceiver, and the control unit.

In some embodiments, the transceiver comprises a wireless transceiver unit for transmitting the continuous image of HbO₂ and the question and answer audio signal wirelessly. In detail, in some embodiments, the wireless transceiver unit is a Bluetooth transceiver, a WiFi transceiver, or a Zigbee transceiver.

In some embodiments, the control unit is electrically connected to the transceiver for receiving an operation command.

In some embodiments, the transceiver comprises a connecting port which can be selectively connected to a cable to transmit the continuous image of HbO₂ and the question and answer audio signal.

In some embodiments, the power supply unit is disposed in the wearable garment, and in other embodiments, the power supply unit is disposed in the control unit.

In some embodiments, the control unit comprises an electrical connector for connecting a commercial power.

In some embodiments, the sound-recording unit is an omnidirectional microphone.

In some embodiments, the question and answer audio signal is uncompressed audio code or compressed audio code.

Based on the above configuration, the test of physiological changes and the test of behavioral ability can be tested in one station with the wearable cognitive function evaluation assisting device. The cooperation of the NIR light source and the image capturing unit would not cause uncomfortable to the respondent, and the all the tools are integrated in the wearable garment. Therefore, the selection of test environment is much easier.

### BRIEF DESCRIPTION OF THE DRAWINGS

FIG. 1 is a perspective view for a first embodiment of the instant disclosure.

FIG. 2 is a block diagram for the first embodiment of the instant disclosure.

FIG. 3 is a partial schematic view in test of test of physiological changes.

FIG. 4 is a schematic view in test of and the test of behavioral ability.

FIG. 5 is a block diagram for the second embodiment of the instant disclosure.

### DETAILED DESCRIPTION

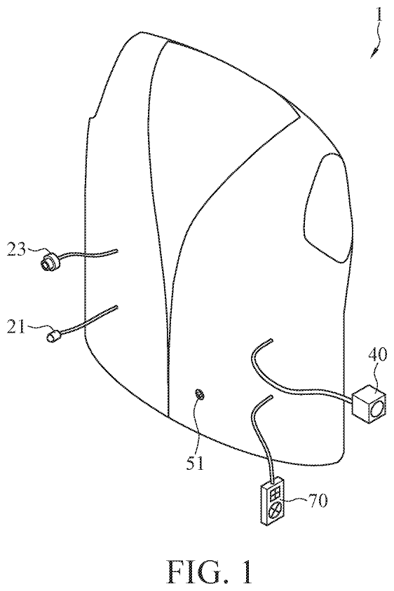

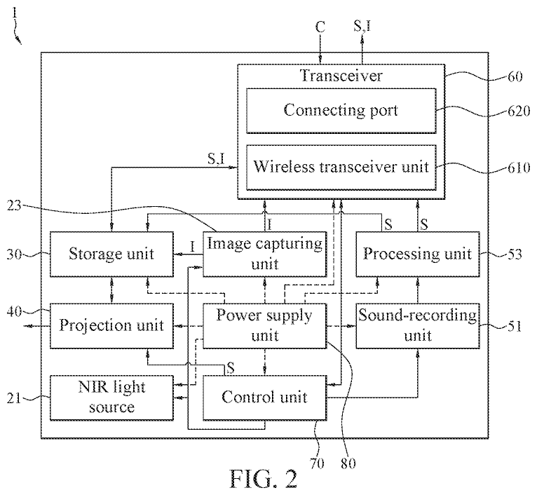

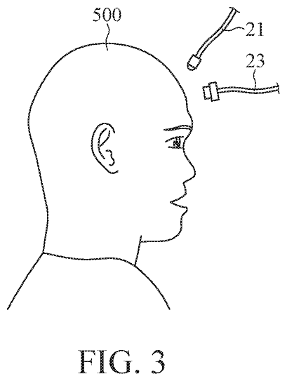

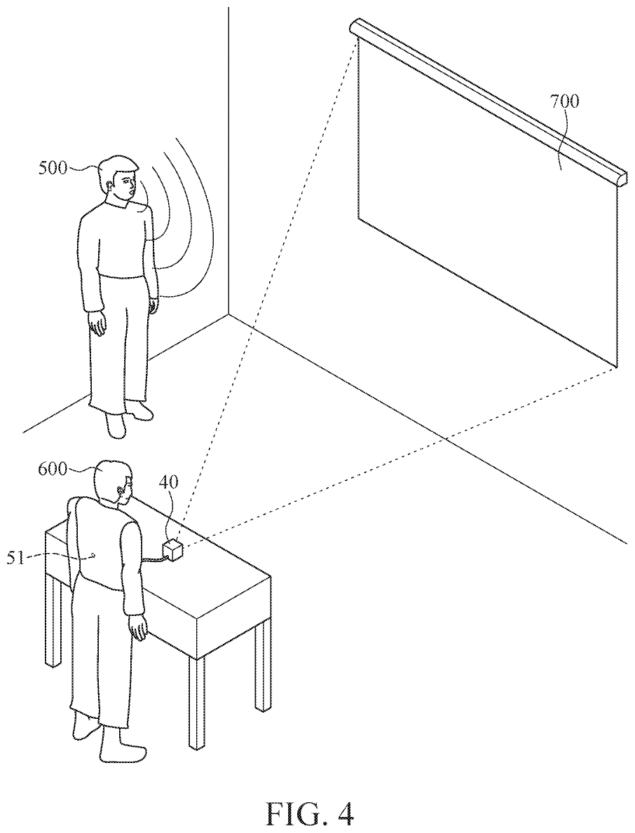

FIG. 1 is a perspective view for a first embodiment of the instant disclosure. FIG. 2 is a block diagram for the first embodiment of the instant disclosure. FIG. 3 is a partial schematic view in test of test of physiological changes. FIG. 4 is a schematic view in test of and the test of behavioral ability. Please refer to FIGS. 1 to 4, a wearable cognitive function evaluation assisting device 1 includes a wearable garment 10, a near infrared (NIR) light source 21, an image capturing unit 23, a storage unit 30, a projection unit 40, a sound-recording unit 51, a processing unit 53, a transceiver 60, a control unit 70, and a power supply unit 80.

As shown in FIGS. 1 to 3, the wearable garment 10 is a wearable clothing material, such as a vest, a jacket, a coat. The NIR light source 21 is disposed on the wearable garment 10, such as connected to the wearable garment 10 through a cable. The NIR light source 21 can be selectively extracted from the wearable garment 10. The image capturing unit 23 is disposed on the wearable garment 10 and can be selectively extracted from the wearable garment 10 for capturing a continuous image of an oxyhemoglobin (HbO₂) where the NIR light source is irradiated. As shown in FIG. 3, during the test of physiological changes, the NIR light source 21 is irradiated on the forehead of a respondent 500, the image capturing unit 23 also focuses on the forehead of the respondent 500 for capturing a continuous image of HbO₂ I of forehead of the respondent 500. Moreover, the image capturing unit 23 can be connected to the storage unit 30, and the capturing a continuous image of HbO₂ I can be stored in the storage unit 30.

As shown in FIGS. 1, 2 and FIG. 4, the storage unit 30 is disposed in the wearable garment 10 and stores a plurality of cognitive environment images. The projection unit 40 is disposed on the wearable garment and electrically connected to the storage unit 30, and can be selectively extracted from the wearable garment 10 for projecting one of the cognitive environment images. The sound-recording unit 51 is disposed on the wearable garment 10 and electrically connected to the storage unit 30 for recording a question and answering sound. The processing unit 53 is disposed in the wearable garment 10 and electrically connected to the sound-recording unit 51 for converting the question and answer sound into a question and answer audio signal S and sending the question and answer audio signal S out. Moreover, the processing unit 53 can be connected to the storage unit 30, and the question and answer audio signal S can be stored in the storage unit 30.

The transceiver 60 is disposed in wearable garment 10 and electrically connected to the image capturing unit 23 and the processing unit 53, the transceiver 60 receives the continuous image of HbO₂ I and the question and answer audio signal S and then transmits the continuous image of HbO₂ I and the question and answer audio signal S out. The control unit 70 is disposed on the wearable garment 10 and electrically connected to turning on and off the NIR light source 21, the image capturing unit 23, the projection unit 40, and the sound-recording unit 51. The power supply unit 80 supplies power to the NIR light source 21, the image capturing unit 23, the storage unit 30, the projection unit 40, the sound-recording unit 51, the processing unit 53, the transceiver 60, and the control unit 70.

In detail, as shown in FIG. 4, during the test of behavioral ability, the host 600 wears the wearable cognitive function evaluation assisting device 1, and controls the projection unit 40 to project a cognitive environment image on a projection medium 700, and ask some questions according to the cognitive environment image. The respondent 500 responds the questions corresponding to the questions. The sound-recording unit 51 records the question and answering sound S. In particular, the sound-recording unit 51 is an omnidirectional microphone which is useful to receive the sounds from multiple directions.

In some embodiments, the processing unit 53 can convert the question and answer sound into a question and answer audio signal S uncompressed audio code (with filename extension of “.flac”, “ape”, or “way”). In other words, the question and answer audio signal S encoded with lossless compressed audio code is not distorted when the audio signal S is sampled. In other embodiments, the processing unit 53 can convert the question and answer sound into a question and answer audio signal S compressed audio code (with filename extension of “.mp3”, “.wma”, or “.ogg”).

As shown in FIG. 2, the transceiver 60 includes a wireless transceiver unit 610 for transmitting the continuous image of HbO₂ I and the question and answer audio signal S out wirelessly. The continuous image of HbO₂ I and the question and answer audio signal S can be sent to a medical cloud terminal, and the specialist can evaluate the cognitive functions of the respondent 500 based on the continuous image of HbO₂ I and the question and answer audio signal S. In detail, the wireless transceiver unit 610 can be a Bluetooth transceiver, a WiFi transceiver, or a Zigbee transceiver.

Moreover, the wireless transceiver unit 610 can further receives operation command C wirelessly from outside, such as a smart phone, a tablet, a laptop computer . . . etc. The control unit 70 is electrically connected to the transceiver 60 for receiving an operation command C, and generates a control signal corresponding to the operation command C. In addition, the question and answer audio signal S and continuous image of HbO₂ I can be stored in the storage unit 30. The transceiver 60 accesses the question and answer audio signal S and continuous image of HbO₂ I based on the control signal from the control unit 70 then sent the question and answer audio signal S and continuous image of HbO₂ I out.

Furthermore, as shown in FIG. 2, the transceiver 60 further comprises a connecting port 620, the connecting port 620 can be selectively connected to a cable to transmit the continuous image of HbO₂ I and the question and answer audio signal S. in the other words, the continuous image of HbO₂ I and the question and answer audio signal S can be transmitted out via a wire or wirelessly. The operation command can also be received via a wire or wirelessly.

As shown in FIG. 2, the power supply unit 80 is disposed in the wearable garment 10. The power supply unit 80 can be implemented by a battery device. The battery device can be charged or replaced easily.

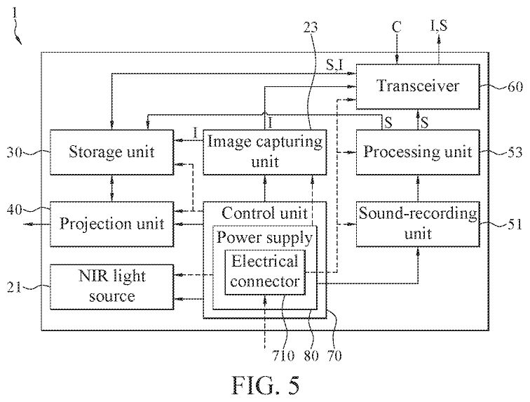

FIG. 5 is a block diagram for the second embodiment of the instant disclosure. As shown in FIG. 5, the power supply unit 80 is disposed in the control unit 70. The power supply unit 80 can be implemented by a battery device, or an AC-DC converter. In some embodiments, the control unit 70 comprises an electrical connector 710 for connecting to a commercial power. The electrical connector 710 connected to the power supply unit 80 for charging the battery, or converting to a DC current to supply the elements in the wearable cognitive function evaluation assisting device 1.

Based on the above configuration, the test of physiological changes and the test of behavioral ability can be tested in one station with the wearable cognitive function evaluation assisting device 1. The cooperation of the NIR light source 21 and the image capturing unit 23 would not cause uncomfortable to the respondent 500, and the all the tools are integrated in the wearable garment 10. Therefore, the selection of test environment is much easier.

While the instant disclosure has been described by way of example and in terms of the preferred embodiments, it is to be understood that the invention needs not be limited to the disclosed embodiments. For anyone skilled in the art, various modifications and improvements within the spirit of the instant disclosure are covered under the scope of the instant disclosure. The covered scope of the instant disclosure is based on the appended claims.

What is claimed is:

1. A wearable cognitive function evaluation assisting device, comprising:
a wearable garment;
a near infrared (NIR) light source disposed on the wearable garment, and being selectively extracted from the wearable garment;
an image capturing unit disposed on the wearable garment and being selectively extracted from the wearable garment for capturing a continuous image of an oxyhemoglobin (HbO₂) where the NIR light source is irradiated;
a storage unit disposed in the wearable garment and storing a plurality of cognitive environment images;
a projection unit disposed on the wearable garment and electrically connected to the storage unit, and being selectively extracted from the wearable garment for projecting one of the cognitive environment images;
a sound-recording unit disposed on the wearable garment and electrically connected to the storage unit for recording a question and answering sound;
a processing unit disposed in the wearable garment and electrically connected to the sound-recording unit for converting the question and answer sound into a question and answer audio signal and sending the question and answer audio signal out;
a transceiver disposed in wearable garment and electrically connected to the image capturing unit and the processing unit for receiving the continuous image of HbO₂ and the question and answer audio signal and then transmitting the continuous image of HbO₂ and the question and answer audio signal out;
a control unit disposed on the wearable garment and electrically connected to turning on and off the NIR light source, the image capturing unit, the projection unit, and the sound-recording unit; and
a power supply unit supplying power to the NIR light source, the image capturing unit, the storage unit, the projection unit, the sound-recording unit, the processing unit, the transceiver, and the control unit.

2. The wearable cognitive function evaluation assisting device of claim 1, wherein the transceiver comprises a wireless transceiver unit for transmitting the continuous image of HbO₂ and the question and answer audio signal wirelessly.

3. The wearable cognitive function evaluation assisting device of claim 2, wherein the wireless transceiver unit is a Bluetooth transceiver, a WiFi transceiver, or a Zigbee transceiver.

4. The wearable cognitive function evaluation assisting device of claim 2, wherein the control unit is electrically connected to the transceiver for receiving an operation command.

5. The wearable cognitive function evaluation assisting device of claim 1, wherein the transceiver comprises a connecting port being selectively connected to a cable to transmit the continuous image of HbO₂ and the question and answer audio signal.

6. The wearable cognitive function evaluation assisting device of claim 1, wherein the power supply unit is disposed in the wearable garment.

7. The wearable cognitive function evaluation assisting device of claim 1, wherein the power supply unit is disposed in the control unit.

8. The wearable cognitive function evaluation assisting device of claim 1, wherein the control unit comprises an electrical connector for connecting a commercial power.

9. The wearable cognitive function evaluation assisting device of claim 1, wherein the sound-recording unit is an omnidirectional microphone.

10. The wearable cognitive function evaluation assisting device of claim 1, wherein the question and answer audio signal is uncompressed audio code or compressed audio code.
